(19) United States
(12) Patent Application Publication
(10) Pub. No.: US 2003/0118535 A1
(43) Pub. Date: Jun. 26, 2003

(54) Depilatory compositions and kits containing the same

(76) Inventors: Adam Lustbader, Chicago, IL (US); Jose Torres, Chicago, IL (US)

Correspondence Address: STEINBERG & RASKIN, P.C., 1140 AVENUE OF THE AMERICAS, 15th FLOOR, NEW YORK, NY, 10036-5803, US

(21) Appl. No.: 09/949,157

(22) Filed: Sep. 7, 2001

(51) Int. Cl. A61K 7/06; A61K 33/00

(52) U.S. Cl. 424/70.1; 424/722

(57) ABSTRACT

A depilatory composition and kit including the same are disclosed. The composition includes an emulsion having a viscosity of less than about 200 cps and an effective amount of a hair removing ingredient such as a thioglycolic acid. Methods of using the same are also disclosed and include spraying an effective amount of the emulsion on to a skin area containing unwanted hair, allowing the emulsion to remain in contact with the hair containing skin for a sufficient amount of time and thereafter wiping the skin to remove the remnants of the emulsion and hair.

### BACKGROUND OF THE INVENTION

1. Field of the Invention

This invention relates to improved compositions and methods for hair removal. In particular, the invention relates to improved sprayable liquid formulations for removing mammalian hair from unwanted areas of the skin.

2. Description of the Related Art

It is common practice among most of the world's population to remove hair in order to improve personal appearance. Women are especially interested in removing unwanted hair from their legs, underarms, face and the like. Shaving with either a razor blade or electric shaver is currently the most frequently employed means of removing hair from these areas. Other means include tweezers, hot wax treatments and depilatory compositions which are applied to the skin in the form of a foam, cream, lotion, gel or mousse. These prior art methods have significant disadvantages such as razor cuts, pain from the tweezers or hot wax, risk of infection, messiness of depilatories together with irritation from such harsh chemicals. The common thread associated with each method is that the hair removal is only temporary. Therefore, the hair removal procedures must be repeated endlessly. Consequently, it would be highly desirable to provide hair removal compositions and methods which are not associated with as many of the problems described above.

Depilatory foams and the like have certain appeal to consumers because of their ability to eliminate the need for using a razor or other apparatus to physically remove the hair from the follicle. In many cases, the active depilatory ingredient of the composition is a thioglycolic acid, such as potassium or calcium thioglycolate. These materials act by penetrating the hair and destroying the cystine bonds between the hair molecules. This weakens the hair to such an extent that the action of scraping and wiping away the depilatory composition causes the hair to break off at skin level, so that the hair is removed. It is also known that certain hair removal formulations are usually aqueous solutions of thioglycolic acid mixed with an alkali such as sodium hydroxide or calcium hydroxide. In addition to breaking disulfide bonds, these aqueous solutions of thioglycolates provide the important function of hydrating the hair shaft. This quickly gives the hair a jelly-like consistency so that it can be more easily wiped away.

A particular problem with many depilatory compositions of this type is that it is difficult to completely remove such compositions from the skin. For example, they cannot be removed by simply rinsing. Often, a heavy or vigorous mechanical scrubbing or scraping action is required, for example with a spatula, which can be a cause of considerable discomfort and inconvenience. Thus, further improvements would be desirable.

Another shortcoming associated with many conventional cream, lotion, gel or mousse depilatory compositions is that the emulsion components are normally stabilized by including a relatively high amount of a thickener. For example, conventional thickeners are generally present at levels of up to 30% w/w. Alternatively, depilatory creams, lotions and gels can be formulated to give a stable emulsion by including high levels of fatty materials in the depilatory composition. In such compositions, either no thickener is present, or a small amount of thickener may be present. In both cases, however, it is difficult to quickly and completely remove the depilatory composition and hair from the skin. In cases where high levels of thickener are employed, there are strong steric interactions between the polymeric materials in the emulsion that it is not possible to remove the depilatory composition quickly and easily from the skin by the mechanical action of water together with a light rubbing action. Where high levels of fatty materials are present, the texture or “body” of the depilatory composition will be too strong to allow the composition to be rinsed simply from the skin. Thus, improvements in emulsion-based compositions which can be more quickly and completely removed from the treated area of the skin are desirous as well.

Hence, those concerned with the development and use of hair removal systems and procedures have long recognized the need for improvements in hair removal compositions, methods and apparatus to enable more rapid, reliable, comfortable, convenient and economical removal of hair. The present invention addresses these needs.

### SUMMARY OF THE INVENTION

In one preferred embodiment of the invention there is provided a depilatory composition containing an emulsion having a viscosity of less than about 200 cps and an effective amount of a hair removing ingredient such as a thioglycolic acid.

In another aspect of the invention, there is provided a method of removing hair from the skin of a mammal. The method includes applying an effective amount of the depilatory composition described above to the skin of a mammal having hair thereon, allowing the depilatory composition to remain in contact with the skin for a sufficient amount of time to substantially remove the hair and thereafter removing the depilatory composition and hair from the skin.

In a still further aspect of the invention there is provided a depilatory kit for removing hair from the skin of a mammal. The kit includes the depilatory composition described above, a container for storing the depilatory composition and a dispenser in communication with the container and capable of dispensing the depilatory composition from the container, preferably in the form of dispersed droplets.

Methods of preparing the depilatory compositions described herein are also disclosed.

For purposes of the present invention, “depilatory” shall be understood to mean a composition capable of removing undesired hair from the body. “Sufficient” shall be understood to mean an amount which is capable of achieving a desired result.

As a result of the present invention there are provided hair removal compositions which can be conveniently sprayed onto skin surfaces to quickly and gently remove unwanted body hair. The unique combination of ingredients included therein allow the skin irritation associated with prior art depilatory products to be substantially reduced or avoided. In preferred aspects, the person using the compositions simply sprays the composition on, waits a short period of time and then wipes away unwanted hair. Moreover, the hair removal achieved is often relatively long lasting. In many instances, the compositions of the present invention provide substantially hair free areas of skin after treatment for up to six weeks or longer.

### DETAILED DESCRIPTION OF THE INVENTION

The depilatory compositions of the present invention are preferably oil in water emulsions which contain not only a hair removing active ingredient but also a combination of additional ingredients which provide long lasting, clean and gentle removal of unwanted hair from the skin. The compositions of the invention, i.e. the emulsions, are preferably dispensed from a spray bottle and thus must have a viscosity which is sufficient to maintain optimum or at least sufficient skin contact time and avoid or substantially minimize run off. The inventive compositions are also capable of substantially freely passing through a finger activated pump sprayer. In accordance therewith, the depilatory compositions of the present invention have a viscosity of less than about 200 cps. In preferred aspects of the invention, the inventive emulsions have a viscosity of from about 75 to about 150 cps. More preferably, the emulsions have a viscosity of from about 90 to about 110 cps.

The compositions of the present invention include at least one hair removing active ingredient. The active ingredient or depilatory agent will preferably act by reducing disulfide bonds in the hair. In principle, any suitable reducing agent may be used. Suitable examples include mercaptans and sulfides such as organic sulfides, especially alkyl sulfides. Preferred depilatory agents are thioglycolic acids or alkaline salts of thioglycolic acid such as the lithium, sodium, potassium or calcium salts. The potassium and calcium salts, alone or in mixture, are preferred. Other suitable depilatory agents include thioglycerol, mercapto propionic acid, dithioerythritol and alkalis such as sodium hydroxide (preferred) or calcium hydroxide. The foregoing is meant to be illustrative and not limiting with regard to the suitable active (depilatory) ingredients. It is intended that all known depilatory compositions be included as being suitable for purposes of the present invention. The active ingredient is present in an amount which is effective for the purpose and will also be described herein as an effective amount which is sufficient to substantially remove the hair from the area being treated. In many aspects, the compositions of the present invention contain from about 0.5 to about 8 percent by weight of the active ingredient(s). In preferred aspects, the amount is from about 2.5 to about 6.5 percent by weight. In a preferred embodiment, the emulsion contains about equal parts by weight thioglycolic acid and sodium hydroxide.

The emulsion-based compositions of the present invention have pH of from about 11.0 to about 13.0, preferably from about 12.25 to about 12.75 and most preferably about 12.5.

In certain aspects, the emulsions preferably include the following ingredients:

| | acceptable range | Preferred |
|---|---|---|
| ingredient | weight % | weight % |
| ceteareth-20 | 1-4 | 0.5-3 |
| emulsifiers | 0.5-8 | 1-2 |
| hair removing | 0.5-8 | 2.5-6.5 |
| active ingredient(s) | | |
| glyceryl monostearate | 0.3-2.0 | 0.4-1.0 |
| mineral oil | 0.5-2.0 | 0.8-1.2 |
| water | at least about 75 | at least about 85 |

It will be understood that any art recognized alternatives to the foregoing may be employed as well. For example, art recognized emulsifiers which are suitable include cetyl alcohol and stearyl alcohol or the combination product cetearyl alcohol.

The inventive compositions can also include optional ingredients which impart one or more desirable and/or therapeutic properties to the final composition. A non-limiting list of such ingredients includes substances such as vitamin C, vitamin E, aloe vera, exfoliants, moisturizers, fragrances, colorants, emolients, etc.

In another aspect of the invention, there is provided a method of removing hair from the skin of a mammal. The method includes applying an effective amount of the depilatory composition described above to the skin of a mammal having hair thereon, allowing the depilatory composition to remain in contact with the skin for a sufficient amount of time to allow it to substantially remove the hair before removing the depilatory composition and loosened hair from the skin. The amount of time the depilatory composition remains in contact on the skin is preferably less than about ten minutes and more preferably is less than about eight minutes. It will be understood by those of ordinary skill that the length of time that will be required will depend on several factors including, but not limited to the amount of hair present on the skin, the amount of the inventive composition applied, the amount of hair removing active ingredient present in the composition, the sensitivity of the skin being treated, etc. The amount of time which is sufficient will be apparent to the artisan of ordinary skill without undue experimentation.

In practice, the method will preferably include the following steps:

1. cleaning the area of the skin where the unwanted hair is located

2. spraying a light mist from the spray bottle containing the inventive composition after shaking the bottle well

3. leaving the mist on the skin for a sufficient amount of time (e.g. about 5 minutes)

4. removing the inventive composition and hair from the skin by wiping it off in a gentle patting motion using a damp cloth; and

5. rinsing the treated area with cool water.

In a still further aspect of the invention there is provided a depilatory kit for removing hair from the skin of a mammal. The kit includes the depilatory composition described above, a container for storing the depilatory composition and a dispenser in communication with the container and capable of dispensing the depilatory composition from the container in the form of dispersed droplets or a mist. In most preferred aspects, the container and dispenser form a pump spray bottle which is capable of dispensing a mist or droplets of the emulsion onto the desired area of the skin.

In order to maximize the amount of skin contact time and minimize run-off of the product from the skin, the inventive liquid must have a ceratin viscosity. However, in order to efficiently dispense the emulsion from the container using a pump spray bottle, care must be taken in selecting the viscosity for the product. It has been surprisingly found that emulsions having a viscosity range described herein, i.e. viscosity of less than about 200 cps, preferably from about 75 to about 150 cps and more preferably from about 90 to about 110 cps, are thick enough to substantially remain in contact with the skin after being atomized through a sprayer yet thin enough to pass through an economical pump sprayer without clogging. In preferred aspects of the invention, the pump sprayer employed has a dip tube diameter of at least about 0.08 inches and preferably about 0.10 inches and has sufficient venting to allow to allow liquids of the viscosity described herein to travel up the dip tube. One preferred pump sprayer is available as the EuroMist HV (available from Seaquist Perfect Dispensing).

Forming the Oil in Water Emulsion

The following procedure is generally carried out to make the depilatory compositions of the present invention. Specific details concerning certain preferred embodiments are provided in the examples.

Generally speaking, standard emulsion techniques are employed. The oil phase ingredients are combined under heated conditions until dissolved. Separately, the water and water soluble components are combined in suitable mixing vessel and the pH is adjusted, if necessary. The melted oil soluble components are then run into the water phase with brisk stirring and then cooled to yield a white, creamy liquid with a viscosity within the range discussed above. The pH is adjusted if necessary to the range discussed above.

### EXAMPLES

The following non-limiting examples illustrate certain aspects of the invention. All parts and percentages are by weight unless otherwise noted and all temperatures are in degrees F. The principal chemicals were obtained from commercial suppliers.

### Example 1

A sprayable hair remover was prepared to contain the following ingredients

| Ingredient | Weight % |
|---|---|
| Glyceryl monostearate | 0.76 |
| Ceteareth 20 | 2.01 |
| cetearyl alcohol | 1.62 |
| mineral oil | 1.62 |
| water | 88.83 |
| thioglycolic acid | 2.77 |
| sodium hydroxide | 2.30 |
| aloe vera extract | 0.0125 |
| vitamin C | 0.00125 |
| vitamin E | 0.0125 |
| fragrance | 0.05 |
| orange oil | 0.0002 |

Step 1. The oil phase was prepared by combining the glyceryl monostearate, cetearyl alcohol and mineral oil, heating the mixture to between 135-140 degrees F. until dissolved.

Step 2. The water phase was prepared by blending the water, thioglycolic acid, sodium hydroxide, aloe vera, vitamin C, vitamin E, fragrance and orange oil. The ingredients were mixed well for about 20 minutes.

Step 3. The water phase and oil phase were combined and mixed for about one hour, initially at about 95 degrees F. and then at 85 degrees F. The pH was tested and adjusted to about 12.5 and, upon cooling to room temperature, the viscosity was found to be about 100 cps.

Step 4. The cooled white liquid was then divided into 4 ounce portions, transferred to spray bottles.

### Example 2

The process of Example 1 is repeated except that the vitamin C and vitamin E are omitted and the weight of the remaining ingredients is adjusted to account for removal.

### Example 3

In this example, the hair remover spray of Example 1 was tested on twenty (20) healthy female subjects, ranging in age from nineteen (19) to fifty four (54) years and who were free from any skin disorder that may effect the test results. The subjects also agreed to not to use any other hair removal product or method during the study period. Subjects currently using any systemic or topical corticosteroid, anti-inflammatory drug or antihistamines on a regular basis, pregnant or lactating, or those with known allergies or sensitivity to hair removal products or those exhibiting irritation, abrasions and lacerations on the legs were excluded.

The candidates for study participation were instructed to refrain from shaving or using hair removal products for one (1) to two (2) weeks prior to irritation of the study. Subjects meeting the above referenced criteria of the study protocol and demonstrating an absence of erythema or irritation on the legs were impaneled. The hair type, amount of hair present and evidence of irritation at baseline were recorded for each subject. Hair type was classified as fine, medium or course. The subject demographics are set froth below in Table I.

TABLE-I
Subject Demographics

| Subject Number | Age | Hair Type | Hair Growth |
|---|---|---|---|
| 1 | 33 | Coarse | Heavy |
| 2 | 42 | Medium/Coarse | Moderate |
| 3 | 44 | Fine/Medium | Moderate |
| 4 | 30 | Medium/Coarse | Moderate |
| 5 | 48 | Fine | Moderate |
| 6 | 54 | Medium | Moderate |
| 7 | 36 | Fine | Moderate |
| 8 | 34 | Fine | Moderate |
| 9 | 41 | Coarse | Moderate |
| 10 | 45 | Medium | Moderate |
| 11 | 41 | Medium | Moderate |
| 12 | 36 | Fine | Moderate |
| 13 | 25 | Medium | Moderate |
| 14 | 19 | Medium | Moderate |
| 15 | 44 | Medium | Moderate |
| 16 | 31 | Medium | Moderate |
| 17 | 41 | Medium | Moderate |
| 18 | 19 | Fine | Slight |
| 19 | 45 | Fine/Medium | Moderate |
| 20 | 41 | Medium/Coarse | Moderate |

Three (3) test sites were defined on the lower legs of each subject. One (1) test site on either the right or the left leg was marked, with a gentian violet pen, to delineate a three (3) inch by six (6) inch area, which was designated for the evaluation of depilation time (Site 1). On the opposite leg, two (2) equal sized test sites were marked for the evaluation of irritation in relation to exposure time (Sites 2 and 3). Assignment of test to the right and the left legs was in accordance with a randomization code.

A technician applied the test material evenly to the site designated for measuring depilation time. Immediately after application of the test material, a timer was set to count from zero (0). One (1) technician was responsible for timing the procedure, while a second technician evaluated the hair removal. At two (2) minutes post-application, a small section of the treated area was wiped with a damp paper towel to determine the extent of hair removal. The exposed area was covered again with the test material. At ten (10) second intervals, different areas of the same test site were checked for depilation, and no area was checked more than once. When the technician observed that the hair was easily removed from the area evaluated, the depilation time, i.e. the time that the test material was on the skin, was recorded. After recording depilation time, the entire test area was covered again with the test material, which remained on the skin for a maximum time of ten (10) minutes and then was rinsed off. The test site was evaluated for irritation immediately after product removal and at ten (10) minutes after removal of the test material.

The test material was applied to each of the two (2) sites on the opposite leg. The test material was allowed to remain on the skin for five (5) minutes on one (1) site and seven (7) minutes for the second site. Subjects were instructed to report any perceptions of sensory irritation while the test material was on the skin of either leg. The test material was removed from the skin for sensory irritation reported as severe. Sites 2 and 3 were evaluated for irritation, immediately after product removal and at ten (1) minutes after removal of the test material. In addition, the subjects were required to return to the test lab at twenty-four (24) hours post-treatment for an additional evaluation of irritation on all three (3) test sites.

Test Results

1. Depilation Time

The depilation time for each subject is listed in Table II.

TABLE-II
Depilation Times

| Subject Number | Depilation Time (Minutes:Seconds) |
|---|---|
| 1 | 6:40 |
| 2 | 7:30 |
| 3 | 3:50 |
| 4 | 8:00 |
| 5 | 4:30 |
| 6 | 4:40 |
| 7 | 5:20 |
| 8 | 5:20 |
| 9 | 6:20 |
| 10 | 6:20 |
| 11 | 4:40 |
| 12 | 4:30 |
| 13 | 6:40 |
| 14 | 5:20 |
| 15 | 5:00 |
| 16 | 5:40 |
| 17 | 6:30 |
| 18 | 3:50 |
| 19 | 8:10 |
| 20 | 5:20 |
| Mean | 5:42 |

Depilation times ranged from three (3) minutes and fifty (50) seconds to eight (8) minutes and ten (10) seconds for all subjects tested. The average depilation time of the rest of the test population was (5) minutes and forty-two (42) seconds.

2. Irritation Evaluations

Irritation scores for the baseline, immediate, ten (10) minute and twenty-four (24) hour evaluations of Sites 1, 2 and 3 appear in Table III. The scoring scale is set forth below.

1.0=No erythema

2.0=Trace erythema

3.0=Mild erythema

4.0=Moderate erythema

5.0=Severe erythema

V=Vesicles, blisters, watery pimples

E=Edema, Swelling

D=Dryness

F=Follicular erythema

S=Scaling

TABLE-III
Irritation Scores

| Sub- | Site 1 (10 Minute Exposure) | | | | Site 2 (7 Minute Exposure) | | | | Site 3 (5 Minute Exposure) | | | |
|---|---|---|---|---|---|---|---|---|---|---|---|---|
| ject | Base | Immedi- | 10 | | Base- | Immedi- | 10 | | | Immedi- | 10 | 24 |
| No. | line | ate | Minutes | 24 Hours | line | ate | Minutes | 24 Hours | Baseline | ate | Minutes | Hours |
| 1 | 1.0 | 1.0 | 1.0 | 1.0 | 1.0 | 1.0 | 1.0 | 1.0 | 1.0 | 1.0 | 1.0 | 1.0 |
| 2 | 1.0 | 1.0 | 1.0 | 1.0 | 1.0 | 1.0 | 1.0 | 1.0 | 1.0 | 1.0 | 1.0 | 1.0 |
| 3 | 1.0 | 1.0 | 2 F | 1.0 | 1.0 | 1.0 | 1.0 | 1.0 | 1.0 | 1.0 | 2.0 | 1.0 |
| 4 | 1.0 | 1.0 | 1.0 | NR | 1.0 | 1.0 | 1.0 | NR | 1.0 | 1.0 | 1.0 | NR |
| 5 | 1.0 | 1.0 | 1.0 | 1.0 | 1.0 | 1.0 | 1.0 | 1.0 | 1.0 | 1.0 | 1.0 | 1.0 |
| 6 | 1.0 | 1.0 | 1.0 | 1.0 | 1.0 | 1.0 | 1.0 | 1.0 | 1.0 | 1.0 | 1.0 | 1.0 |
| 7 | 1.0 | 1.0 | 1.0 | 1.0 | 1.0 | 1.0 | 1.0 | 1.0 | 1.0 | 1.0 | 1.0 | 1.0 |
| 8 | 1.0 | 1.0 | 1.0 | 1.0 | 1.0 | 1.0 | 1.0 | 1.0 | 1.0 | 1.0 | 1.0 | 1.0 |
| 9 | 1.0 | 1.0 | 3.0 | 4.0 F | 1.0 | 1.0 | 3.0 | 2.0 | 1.0 | 1.0 | 4.0 | 2.0 |
| 10 | 1.0 | 1.0 | 1.0 | 1.0 | 1.0 | 1.0 | 1.0 | 1.0 | 1.0 | 1.0 | 1.0 | 1.0 |
| 11 | 1.0 | 1.0 | 1.0 | 1.0 | 1.0 | 1.0 | 1.0 | 1.0 | 1.0 | 1.0 | 1.0 | 1.0 |
| 12 | 1.0 | 1.0 | 1.0 | 1.0 | 1.0 | 1.0 | 1.0 | 1.0 | 1.0 | 1.0 | 1.0 | 1.0 |
| 13 | 1.0 | 1.0 | 2.0 | 3.5 F | 1.0 | 1.0 | 1.0 | 1.0 | 1.0 | 1.0 | 1.0 | 1.0 |
| 14 | 1.0 | 1.0 | 1.0 | 1.0 | 1.0 | 1.0 | 1.0 | 1.0 | 1.0 | 1.0 | 1.0 | 1.0 |
| 15 | 1.0 | 1.0 | 1.0 | 1.0 | 1.0 | 1.0 | 1.0 | 1.0 | 1.0 | 1.0 | 1.0 | 1.0 |
| 16 | 1.0 | 1.0 | 1.0 | 2.0 | 1.0 | 1.0 | 1.0 | 2.0 | 1.0 | 1.0 | 1.0 | 1.0 |
| 17 | 1.0 | 2.0 | 3.0 | 2.0 | 1.0 | 1.0 | 1.0 | 1.0 | 1.0 | 1.0 | 1.0 | 1.0 |
| 18 | 1.0 | 1.0 | 1.0 | 1.0 | 1.0 | 1.0 | 1.0 | 1.0 | 1.0 | 1.0 | 1.0 | 1.0 |
| 19 | 1.0 | 1.0 | 2.0 | 2.0 | 1.0 | 1.0 | 2.0 | 2.0 | 1.0 | 1.0 | 2.0 | 2.0 |
| 20 | 1.0 | 3.0 F | 3.5 | 3.0 | 1.0 | 2.0 | 3.5 F | 4.0 | 1.0 | 2.0 | 1.5 | 1.0 |

NR: No Reading. Subject returned at forty eight (48) hours, and no irritation was observed.

Sites exposed to the test material for a ten (10) minute period exhibited erythema ranging from trace to moderate at intervals following applications in seven (7) subjects. The frequency of irritation was lower for sites exposed to the test material for seven (7) minutes and five (5) minutes. Four (4) subjects exhibited trace to moderate erythema on the sites exposed for seven (7) minutes, and four (4) subjects exhibited trace to moderate erythema on the sites exposed for five (5) minutes.

Five (5) subjects reported sensory irritation during the study, which included reports of itching, stinging and burning. For only one (1) of these subjects, the test material was removed from the skin of Site 1 due to severity of the reaction. All the other subjects reported only mild or moderate sensations.

### CONCLUSION

It can be seen from the foregoing that the products of the present invention are generally well tolerated for most subjects. The product tested demonstrated depilation times ranging from five (5) minutes and fifty (50) seconds to nine (9) minutes and forty (40) seconds, with an average time of seven (7) minutes and forty-five (45) seconds measured for the study population. Skin sites exposed to the test material for five (5) minutes, seven (7) minutes and ten (10) minutes demonstrated similar levels and frequency of erythema in the studied population. Incidences of follicular erythema were much more common in the sites exposed to the test material for a ten (10) minute period, relative to sites exposed for seven (7) or five (5) minutes. These irritation levels observed on all test sites are consistent with that expected of the product category of the test material. Subjective reports of irritation were also typical of depilatory products, and a severe reaction was reported by only one (1) subject at seven (7) minutes and thirty (30) seconds of exposure. All subjects demonstrated complete hair removal in approximately ten (10) minutes of test material exposure. However, an increase in follicular erythema was observed after a ten (10) minute exposure time, compared to a seven (7) or five (5) minute exposure which is more likely to be the normal time the inventive compositions are applied to the skin.

Accordingly, it will be apparent from the foregoing that, while particular forms of the invention have been described, various modifications can be made without departing from the spirit and scope of the invention. Therefore, it is not intended that the invention be limited, except as by the appended claims.

What is claimed is:

1. A depilatory composition, comprising an emulsion having a viscosity of less than about 200 cps and an effective amount of a hair removing ingredient.

2. The depilatory composition of claim 1, where said hair removing ingredient comprises a member of the group consisting of thioglycolic acids, sodium hydroxide and combinations thereof.

3. The depilatory composition of claim 1, wherein said emulsion has a viscosity of from about 75 to about 150 cps.

4. The depilatory composition of claim 3, wherein said emulsion has a viscosity of from about 90 to about 110 cps.

5. The depilatory composition of claim 1, wherein said emulsion is an oil in water emulsion.

6. The depilatory composition of claim 5, wherein said oil in water emulsion comprises:

| ingredient | range (wt %) |
|---|---|
| ceteareth-20 | 1-4 |
| emulsifiers | 0.5-8 |
| hair removing | 0.5-8 |
| active ingredient(s) | |
| glyceryl monostearate | 0.3-2.0 |
| mineral oil | 0.5-2.0 |
| water | at least about 75 |

7. The depilatory composition of claim 5, wherein said oil in water emulsion comprises:

| ingredient | ingredient |
|---|---|
| ceteareth-20 | 0.5-3 |
| emulsifiers | 1-2 |
| hair removing | 2.5-6.5 |
| active ingredient(s) | |
| glyceryl monostearate | 0.4-1.0 |
| mineral oil | 0.8-1.2 |
| water | at least about 85 |

8. The depilatory composition of claim 5, wherein said oil in water emulsion comprises:

| Ingredient | Weight % |
|---|---|
| Glyceryl monostearate | 0.76 |
| Ceteareth 20 | 2.01 |
| cetearyl alcohol | 1.62 |
| mineral oil | 1.62 |
| water | 88.83 |
| thioglycolic acid | 2.77 |
| sodium hydroxide | 2.30 |
| aloe vera extract | 0.0125 |
| vitamin C | 0.00125 |
| vitamin E | 0.0125 |
| fragrance | 0.05 |
| orange oil | 0.0002 |

.

9. The depilatory composition of claim 1, wherein said emulsion has a pH of from about 11.0 to about 13.0.

10. The depilatory composition of claim 9, wherein said emulsion has a pH of from about 12.25 to about 12.75.

11. The depilatory composition of claim 10, wherein said emulsion has a pH of about 12.5.

12. A method of removing hair from the skin of a mammal, comprising applying an effective amount of the depilatory composition of claim 1 to the skin of a mammal having hair thereon, allowing said depilatory composition to remain in contact with said skin for a sufficient amount of time to substantially remove the hair present on said skin and thereafter removing said depilatory composition and hair from said skin.

13. The method of claim 12, wherein the time said depilatory composition remains in contact on said skin is less than about ten minutes.

14. The method of claim 13, wherein the time said depilatory composition remains in contact on said skin is less than about eight minutes.

15. A depilatory kit for removing hair from the skin of a mammal, comprising:
a) a depilatory composition, comprising an emulsion having a viscosity of less than about 200 cps and an effective amount of a hair removing ingredient;
b) a container for storing said depilatory composition; and
c) a dispenser in communication with said container and capable of dispensing said depilatory composition from said container in the form of dispersed droplets.

16. The depilatory kit of claim 15, wherein said container and said dispenser form a spray bottle.

17. The depilatory kit of claim 15, wherein said emulsion has a viscosity of from about 75 to about 150 cps.

18. The depilatory kit of claim 17, wherein said emulsion has a viscosity of from about 90 to about 110 cps.

19. The depilatory kit of claim 15, wherein said emulsion contains:

| ingredient | range (wt %) |
|---|---|
| ceteareth-20 | 1-4 |
| emulsifiers | 0.5-8 |
| hair removing | 0.5-8 |
| active ingredient(s) | |
| glyceryl monostearate | 0.3-2.0 |
| mineral oil | 0.5-2.0 |
| water | at least about 75 |

20. The depilatory kit of claim 15, wherein said emulsion has a pH of from about 11.0 to about 13.0.

21. A method of preparing a depilatory composition, comprising forming an oil in water emulsion containing an effective amount of a hair removing ingredient and said emulsion having a viscosity of less than about 200 cps.
